(19) United States
(12) Patent Application Publication
(10) Pub. No.: US 2008/0113441 A1
(43) Pub. Date: May 15, 2008

(54) Isobaric labeling and methods of use thereof

(76) Inventors: Ron Orlando, Athens, GA (US); William S. York, Watkinsville, GA (US); James A. Atwood III, Duluth, GA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 11/985,161

(22) Filed: Nov. 14, 2007

Related U.S. Application Data
(60) Provisional application No. 60/858,783, filed on Nov. 14, 2006.

(51) Int. Cl. G01N 33/58; G01N 33/68; G01N 33/92; G01N 24/00

(52) U.S. Cl. 436/71; 436/86; 436/94; 436/173

(57) ABSTRACT

Briefly described, embodiments of this disclosure include method of identifying compounds and the like.

### CROSS-REFERENCE TO RELATED APPLICATION

This application claims priority to U.S. provisional application entitled, “ISOBARIC LABELING AND METHODS OF USE THEREOF,” having Ser. No. 60/858,783, filed on Nov. 14, 2006, which is entirely incorporated herein by reference.

### STATEMENT REGARDING FEDERALLY SPONSORED RESEARCH OR DEVELOPMENT

This invention was made with Government support under Contract/Grant No. RR018502, awarded by the National Institute of Health. The Government has certain rights in this invention.

### BACKGROUND

Glycosylation is one of the most common post-translational modifications encountered in eukaryotic systems. It has been estimated that 60-90% of all mammalian proteins are glycosylated at some point during their existence. Glycoprotein glycans are essential and often play critical roles in numerous biological systems. Identifying specific glycan structures, deciphering the proteins that express each glycan, and understanding in detail how these structures change, e.g., as cells differentiate or as tumor cells progress are components of the emerging field of glycoproteomics. A large number of proteins are involved in regulating glycan expression and function, including glycosyltransferases, glycosidases, other enzymes involved in sugar nucleotide metabolism and transport, as well as carbohydrate binding proteins known as lectins. The genes that encode many of these enzymes have been isolated, expressed, and characterized extensively by functional studies, including generating null mice. It is estimated that the murine glycome, for example, encodes over 650 genes that effect glycan structure. A major challenge, therefore, is to determine how glycan structures change during progression, how transcripts of genes in the glycome change as cells initiate differentiation programs, and then to synthesize an understanding of how transcript changes can be used to identify and predict changes in glycan expression. A sensitive, quantitative technique for glycoprotein glycan analysis, therefore, is a critical component to this field.

The requirements of a methodology for quantitative glycan expression comparison are its capability of detecting subtle changes in structure. Mass spectrometric techniques based on electrospray ionization (ESI-MS) or matrix assisted laser desorption ionization with time of flight detection (MALDI-TOF-MS) have found important applications in high-throughput proteomic analyses due to substantial improvements in the instrumentation and the development of computer algorithms that allow the analysis of large amounts of data. Studies have shown that it is often possible to detect glycans released from glycoproteins using similar MS techniques without derivatization. However, derivatization of oligosaccharides by permethylation is usually performed before MS analysis, because this chemical modification stabilizes the sialic acid residues in acidic oligosaccharides. Permethylated glycans ionize more efficiently than their native counterparts. Moreover due to their hydrophobic nature, methylated glycans are easily separated from salts and other impurities that may affect the MS analysis. Additionally, the fragmentation of methylated glycans is more predictable than that of their native counterparts, leading to accurate structural assignments when MS/MS analysis are performed.

One means of obtaining quantitative proteomic data from mass spectrometric analyses is through the incorporation of isotopic labels into a population of molecules. In this approach, the sample containing the “heavy” isotope is mixed with the sample containing the “light” isotope, followed by MS analysis of the resulting mixture. The mass analyzer resolves the isotopically labeled species, permitting their relative abundances to be determined from the ratio of the light and heavy molecular ions. Numerous isotopic labeling procedures have been established for the study of protein mixtures and these are widely used in high throughput proteomic studies.

The use of isotopic labels for the quantitative analysis of glycans offers promise for the detection and measurement of changes in the abundance of specific oligosaccharide structures that are present in complex glycoprotein mixtures obtained from cells or tissues. Initial reports have focused on the use of heavy/light methyl iodide [either ¹³CH₃I or ¹²CD₃I with ¹²CH₃₁I] methyl an isotopic label introduced by permethylation reactions prior to MS analysis. While this approach shows promise, there are some limitations. In particular the mass difference between the heavy and light pairs is variable and can be very large since the delta mass between the light and heavy isotopemers increase with the number of hydroxyl groups on the glycan. In addition, this variability can lead to confusion in the analysis of complex mixtures since it can be difficult to match the isotopic pairs. Furthermore, this approach cannot be used for the relative quantification of individual components of the isomeric mixtures often associated with glycomic analyses. To date, the use of isotopic labeling has not gained widespread use in the field of glycomics.

### SUMMARY

Briefly described, embodiments of this disclosure include-method of quantifying compounds and the like. One exemplary method of quantifying compounds, among others, includes: labeling a first set of compounds with a first isobaric label; labeling a second set of compounds with a second isobaric label; mixing the first set of compounds with a second set of compounds to form a third set of compounds; and analyzing the third set of compounds with a mass spectrometry system.

One exemplary method of quantifying compounds, among others, includes: labeling a first set of glycans with a first isobaric label; labeling a second set of glycans with a second isobaric label; mixing the first set of glycans with a second set of glycans to form a third set of glycans; and analyzing the third set of glycans with a mass spectrometry system.

One exemplary method of quantifying compounds, among others, includes: labeling a first set of compounds with a first isobaric label; labeling a second set of compounds with a second isobaric label; labeling at least one more set of compounds with at least one isobaric label, wherein each of the additional sets of compounds is labeled with a unique isobaric label; mixing the first set of compounds, the second set of compounds, and the one or more sets of compounds to form a resultant set of compounds; and analyzing the resultant set of compounds with a mass spectrometry system.

### DRAWINGS

Many aspects of this disclosure can be better understood with reference to the following drawings. The components in the drawings are not necessarily to scale. Moreover, in the drawings, like reference numerals designate corresponding parts throughout the several views.

The patent or application file contains at least one drawing executed in color. Copies of this patent or patent application publication with color drawing(s) will be provided by the Office upon request and payment of the necessary fee.

FIGS. 1a and 1b illustrate flow charts for quantitative analysis using isobaric labeling and quantitative glycan analysis using isobaric labeling, respectively.

FIG. 2 illustrates a FTICR spectra of the triantennary glycan permethylated with (FIG. 2a) ¹³CH₃I and (FIG. 2b) ¹²CH₂DI. FIG. 2c illustrates a FTICR spectrum of a 1:1 mixture of the ¹³CH₃ and ¹²CH₂D labeled fetuin glycan.

FIG. 3 illustrates mass spectra of the quantitation of human serum glycans by QUIBL. Glycans from human serum were permethylated in either ¹³CH₃I or ¹²CH₂DI. The two labeled glycan samples were mixed together at a ratio of 1:1.6 and analyzed using an LTQ-FT in triplicate. FIG. 3a illustrates an ion trap MS spectrum of the serum glycan mixture. The differentially labeled glycan precursor ions appear at the same nominal m/z. FIG. 3b illustrates an FTICR spectrum of a biantennary serum glycan. The calculated expression ration 0.62 corresponds well with the expected ratio for quantitation of the glycan precursor ion. FIG. 3c illustrates the biantennary complex glycan was subjected to MS² in the ion trap and the most abundant ion at 1628.73 m/z was analyzed by FTICR. FIG. 3d illustrates an FTICR spectrum of the glycan fragment ion at 1628.73 m/z. FIG. 3e illustrates MS³ spectrum resulting from collision induced dissociation of MS² fragment ion 1628.73 m/z. FIG. 3f illustrates FTICR spectrum of the MS³ fragment ion at 1158.36 m/z.

FIG. 4 illustrates a correlation between calculated and expected ratios for quantitation of two fetuin glycans by QUIBL. In each experiment the ¹³CH₃ and ¹²CH₂D labeled glycans were mixed together at the ratios 10:1, 8:3, 1:1, 3:8, and 1:10 (¹³CH₃:¹²CH₂D) and analyzed by FTICR. The calculated expression ratios were determined by comparing the sum of the peak intensities for all isotopes between ¹³CH₃ and ¹²CH₂D labeled precursor ions for each glycan. For both glycans a linear correlation was observed between the calculated and the expected ratios with a minimum R² of 0.9983.

FIG. 5 illustrates a QUIBL analysis of a differently labeled fetuin glycan mixed at five different ratios. Two fetuin glycan mixtures were permethylated in either ¹³CH₃I or ¹²CH₂DI. The two differentially labeled glycan mixtures were then mixed together at the ratios 10:1, 8:3, 1:1, 3:8, and 1:10 (¹³CH₃:¹²CH₂D) and analyzed by FTICR (a, b, c, d, e). Accurate quantitation was achieved at all ratios over two orders of magnitude.

FIG. 6 illustrates MSⁿ analysis of two di-fucosylated (Lewis X type) N-linked glycans from ES and EB cells. FIGS. 6a and 6b illustrate MS² of the two Lewis X type N-linked glycans. FIG. 6c illustrates MS³ of the fragment ion at 1846.00 m/z from MS² of the glycan in shown in FIG. 6a. FIG. 6d illustrates MS³ of the fragment ion at 1126.18 m/z from MS² of the glycan in shown in FIG. 6b.

FIGS. 7a-7d illustrates Tables 1-4.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of mass spectrometry, chemistry, organic chemistry, inorganic chemistry, material science, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight or stoichiometries by weight/volume (w/v) or volume/volume (v/v), temperature is in ° C., and pressure is in atmosphere. Standard temperature and pressure are defined as 25° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Discussion

Embodiments of the present disclosure include methods of quantitative glycomics using isobaric labels, methods of using isobaric labeling to analyze compounds, methods of using isobaric labeling to analyze glycans, and the like. In addition, embodiments of the present disclosure include methods of quantitative glycoproteomics and proteomics using isobaric labeling.

The term “isobar” can be defined as one of two or more atoms that have a common mass number but have different atomic numbers. Isobars possess approximately equal masses, but differ in their exact masses. In other words, an isobaric pair is a pair of elements/compounds with the same mass number (thus roughly the same mass) with different ratios of neutrons and protons. It should be noted that embodiments of the present disclosure include the use of isobaric groups (e.g., 3 to 10 isobars), and embodiments are not limited to using only isobaric pairs. Portions of the disclosure describe using isobaric pairs, but those descriptions are provided to simplify the discussion, and using isobaric groups can be applied accordingly to methods of the present disclosure.

Embodiments of the present disclosure can use of isobaric compounds in which the molecular mass difference is resovable by a high resolution mass spectrometry system, but the nominal mass is identical. Embodiments of the present disclosure include elements that can be used to form isobaric pairs or groups and these elements include, but are not limited to, 1/2H, 15/14 N, 13/12 C, 16/18 O, 32/34 S, 35/37 Cl, and 79/81 Br. For example, 14C and 14N are isobars. In another illustrative example, ¹³CH₃I and ¹²CH₂DI are isobaric compounds. Exemplar isobaric compound group can include ¹²C¹⁵NH₅:¹²C¹⁵NH₅: ¹²C¹⁴NH₄D. Another exemplar isobaric compound group can include ¹²C₃H₃¹⁸O₂³²S³⁵Cl₂: ¹²C₃H₃¹⁶O₂³⁴S³⁵Cl₂: ¹²C₃H₃¹⁶O₂³²S³⁷Cl₂: ¹³C₂ ¹²C₁H₃¹⁶O₂³²S³⁵Cl₂: ¹²C₃HD₂¹⁸O₂ ³²S³⁵Cl₂. Thus, a variety of isobaric pairs or groups may be used in embodiments of the present disclosure. In this regard, isobars can be included in compounds to form isobaric labels that have the same nominal mass, but differ in their exact mass. In this regard, various combinations of isobars can be constructed so that the isobar labels have the same nominal mass, but differ in their exact mass. Two or more isobaric labels can be used to perform quantitative analysis on compounds such as, but not limited to, glycans, glycoproteins, proteins, carbohydrates, glycolipids, and the like.

The quantitative analysis is performed using mass spectrometry systems such as, but not limited to, ion trap mass spectrometry systems, linear ion trap mass spectrometry systems, quadrupole mass spectrometry systems, ion cyclotron resonance mass spectrometry systems, time of flight mass spectrometry systems, orbitrap spectrometry systems, and combinations thereof. The mass spectrometry system source can include sources such as, but not limited to, electrospray ionization sources, atmospheric pressure chemical ionization sources, inductively coupled plasma ion sources, glow discharge ion sources, electron impact ion sources, laser desorption/ionization ion sources, radioactive sources, as well as other ion sources compatible with the mass spectrometry systems mentioned above.

Embodiments of the present disclosure include a number of advantages. Many of the advantages derive from the fact that the isobaric ions appear at the same nominal mass to charge ratio. This characteristic leads to increased ion intensity since ions from both samples are not distributed between isotopic species having different m/z values. In addition, the small mass difference between these isobars allows the two species to be simultaneously selected for MSⁿ analysis, which permits the relative quantitation of isomeric compounds. The use of isobaric labeling also minimizes the effects caused by isotopes of the light and heavy species appearing at the same m/z value (described in more detail in Example 1) since the isobars can be resolved by the mass spectrometer. This factor is expected to improve the linear dynamic range and reduce the effects associated with isotopic impurity. For example, embodiments of the present disclosure provide relative quantitative data with a linear dynamic range of at least two orders of magnitude. This procedure was successfully used to analyze N-linked oligosaccharides released from a standard glycoprotein and from human serum as shown in the Example. This strategy is directly applicable to oligosaccharides from other sources, such as glycolipids.

In an embodiment, a group of compounds (e.g., glycans) are divided into a first set of compounds and a second set of compounds. The first set of compounds is mixed and reacted with a first isobaric label so that a plurality of isobaric labels bond to each of the compounds. The second set of compounds is mixed and reacted with a second isobaric label so that a plurality of isobaric labels bond to the compounds. Subsequently, the first set and the second set of compounds are mixed to form a third set of compounds. The third set of compounds is analyzed using one or more mass spectrometry systems to determine mass and structural information about the compounds. In particular, qualitative and relative quantitative data can be obtained about the compounds. Interpretation of the qualitative and relative quantitative data is described in more detail in Example 1.

In an embodiment, methods of the present disclosure are used in isobaric labeling for quantitative glycomics. In general, two sets of glycans are permethylated with either ¹³CH₃I or ¹²CH₂DI. This pair of reagents has the same nominal mass, but differ in their exact mass by 0.002922 Da per label. Glycans typical contain multiple hydroxyl groups (e.g., sites of methylation), which increase the mass difference between the two samples and allows them to be separated with a more modest resolution of about 30,000 m/□m. Since the number of hydroxyl groups increases with the mass of the glycan, the difference between these isobaric species also increases and thus the resolution needs are approximately independent of the glycan's size for typical N- and O-linked species. Additional details regarding embodiments of the disclosure are described in the attached Example 1.

### EXAMPLE

Now having described the embodiments of the present disclosure, in general, Example 1 describes some additional embodiments of the present disclosure. While embodiments of the present disclosure are described in connection with Example 1 and the corresponding text and figures, there is no intent to limit embodiments of the present disclosure to these descriptions. On the contrary, the intent is to cover all alternatives, modifications, and equivalents included within the spirit and scope of embodiments of the present disclosure.

### Example 1

The study of glycosylation patterns (glycomics) in biological samples is an emerging field that can provide key insights into cell development and pathology. A current challenge in the field of glycomics is to determine how to quantify changes in glycan expression between different cells, tissues, or biological fluids. This example describes Quantitation by Isobaric Labeling (QUIBL), which facilitates comparative glycomics. Permethylation of a glycan with ¹³CH₃I or ¹²CH₂DI generates a pair of isobaric derivatives, which have the same nominal mass. However, each methylation site introduces a mass difference of 0.002922 Da. As glycans have multiple methylation sites, the total mass difference for the isobaric pair allows separation and quantitation at a resolution of ˜30,000 m/□m. N-linked oligosaccharides from a standard glycoprotein and human serum were used to demonstrate that QUIBL facilitates relative quantitation over a linear dynamic range of two orders of magnitude and permits the relative quantitation of isomeric glycans. QUIBL was applied to quantitate glycomic changes associated with the differentiation of murine embryonic stem cells to embryoid bodies. Based on these results, QUIBL will be useful for glycomic studies and that this labeling approach may be adapted to other types of “-omic” investigation.

### EXPERIMENTAL

Materials

Bovine fetuin and human blood serum were purchased from Sigma. 99% ¹³CH₃I and 98% CH₂DI were purchased from Cambridge Isotopes Inc (Andover, Mass.). Acetonitrile for chromatography was purchased from Fischer Scientific. Aurum serum protein mini kit for albumin and IgG depletion was purchased from BIORAD.

Cell Culture and Embryoid Body Differentiation

Murine embryonic stem cells (ES) were cultured as previously described (Oncogene 2002, 21, 8320-8333, which is incorporated herein by reference). The ES cell culture media was composed of Dulbecco's modified Eagle's medium (DMEM) supplemented with 10% fetal calf serum (FCS, Commonwealth Serum Laboratories), 1 mM L-glutamine, 0.1 mM 2-mercaptoethanol, and 1000 U/ml recombinant murine leukemia inhibitory factor (LIF) (ESGRO, Chemicon International). The ES cells were cultured at 37° C. under 10% CO₂. ES cells were differentiated into embryoid bodies as previously described (J Cell Sci 2000, 113, (Pt 3), 555-566, which is incorporated herein by reference). ES were first harvested by trypsinization then seeded into 10 cm bacteriological dishes at a density of 1×10⁵ cells/ml, in 10 ml of ES medium lacking LIF. EBs were harvested daily, the media was changed every 2 days, and the cultures were split one into two at day 4. For the glycan analysis, 1×10⁷ ESCs and 1×10⁷ EBs were collected by trypsinization, placed into a 15 ml conical tube, and pelleted at 1,000 g. The cells were washed 3 times in ice cold phosphate buffered saline (PBS) followed by centrifugation at 1000 g after each wash. All supernatant was removed from the tube and the cell pellets were stored at −80° C. until analysis.

ES and EB Cell Lysis and Delipidation

The ES and EB cell lysis was performed by adding 2 mL of water to each cell pellet, placing them into an ice bath, and sonicating for 40 seconds (in four pulses of 10 seconds each) using a probe sonicator at an intensity of 15 watts. Lipids were then extracted from the cells using a modification of the procedure by Svenerholm and Fredman (Biochimica et Biophysica Acta (BBA)—Lipids and Lipid Metabolism 1980, 617, (1), 97-109, which is incorporated by reference). Chloroform and methanol were then added to a final proportion of 4:8:3 (chloroform:methanol:water). The resulting mixture was incubated 2 hours at −20° C. and then water was added to modify the chloroform:methanol:water proportion to 4:8:5.6. The mixture was then centrifuged at 5000 g to separate the three phases. The lower (chloroform rich) and upper (aqueous) phases were carefully removed with a Pasteur pipette and the intermediate layer (protein rich) was added to 1 mL of acetone and centrifuged at 5000 g. The acetone supernatant was removed and the delipidated protein pellet was washed once more with cold acetone, suspended in 2 ml of water, and sonicated as described above. The protein mixture was then lyophilized to dryness.

Human Serum Albumin and IgG Depletion

Albumin and IgG were removed from 100 μl of human serum (Sigma) per the manufacturers' recommendations by passage through a spin column containing Affi-Gel Blue and Affi-Gel protein A.

Protein Digestion and N-Linked Glycan Release

Enzymatic protein digestion and N-linked glycan release was carried out as previously described with minor modifications (J. Proteome Res 2006, 5, (12), 3376-3384, which is incorporated by reference). For the bovine fetuin (100 μg) and the human serum glycoproteins, disulfide bond reduction was first performed by the addition of 40 mM dithiothreitol (DTT) in 50 mM ammonium bicarbonate and incubation at 55° C. for 1 h. Carboxyamidomethylation was then performed by addition of 100 mM iodoacetamide (IDA) in 50 mM ammonium bicarbonate and incubation for 1 h at room temperature in the dark. Samples were then digested overnight at 37° C. with 2 μg TPCK-treated trypsin in 50 mM ammonium bicarbonate buffer. The trypsin was then removed by filtration through a 30 kDa MW cutoff filter (Millipore, Billerica, Mass.) and eluent was collected.

The ES and EB protein pellets were solubilized by the addition of 1 mL of 50 mM Tris and 2 M Urea, pH 8.5 followed by sonication. The proteins were reduced with 25 mM DTT for 45 min at 50° C. and then carbamidomethylated with 90 mM iodoacetamide over 1 hr at room temperature in the dark. Proteolytic digestion was performed overnight at 37° C. in the presence of 100 μg of TPCK-treated trypsin. The resulting mixture of peptides and glycopeptides was desalted using a Sephadex G-15 column (1×50 cm), eluted isocratically with 20 mM ammonium bicarbonate. The desalted peptides/glycopeptides were frozen and lyophilized to dryness.

The N-linked glycans from fetuin, serum, ES, and EB cells were then released by overnight incubation with Peptide: N-Glycosidase F (PNGase F, New England BioLab, 1000 U for serum and fetuin and 3000 U for ES and EB) at 37° C.

Glycan Isolation

Glycans were separated from peptides by reverse phase liquid chromatography. PNGase F digests were loaded onto a C18-Sep-Pak (Waters Corp.), which had been pre-equilibrate in 3% acetic acid and the glycans were eluted from the column by the addition of 4 mL of 3% acetic acid. The fetuin, serum, ES and EB glycans were each divided into two equal aliquots. All of the glycan samples were frozen and lyophilized to dryness.

Glycan Permethylation

Dried glycans (30 μg aliquots) were permethylated as described previously (Glycobiology 2007). Glycans were suspended in DMSO (00.1 mL) and NaOH (20 mg in 0.1 mL of dry DMSO) was added. After strong mixing, 0.1 mL of ¹³CH₃I or ¹²CH₂DI was added. After 10 minutes incubation in a bath sonicator, 1 mL of water was added, and the excess of methyl iodide was removed by bubbling with a stream of N₂. One mL of methylene chloride was added with vigorous mixing, and after phase separation the upper aqueous layer was removed and discarded. The organic phase was then extracted three times with water. Methylene chloride was evaporated under a stream of N₂, and the methylated glycans were dissolved in 25-50 μL of 50% methanol.

Preparation of Glycans for Ms Analysis

The permethylated glycan samples were dissolved in 50% MeOH and 1 mM NaOH for analysis by tandem mass spectrometry. The ¹³CH₃ and ¹²CH₂D labeled glycans from fetuin were first analyzed independently. To determine the dynamic range for QUIBL, the following mixtures of permethylated fetuin glycans were prepared: 10:1, 8:3, 1:1, 3:8, and 1:10 for the ¹³CH₃ to CH₂D. Each mixture was analyzed independently, in triplicate. The ¹³CH₃ and CH₂D labeled serum glycans were mixed at a ratio of 1:1.66 (¹³CH₃:CH₂D). Quantitation of the permethylated glycan mixtures from ESCs and EBs were normalized to the Man₅ structure.

MS Analysis of the Permethylated Glycans

The glycans were analyzed on a hybrid linear ion trap Fourier transform ion cyclotron resonance mass spectrometer (LTQ-FT, Thermo Scientific). Each glycan mixture was infused into the LTQ-FT at a flow rate of 0.3 μl/min and electrosprayed through a 15 μm pulled silica capillary (New Objective, Woburn, Mass.) at 1.9 kV. MSⁿ experiments in the LTQ were carried out in positive ion and profile mode using a normalized collision energy of 29%, activation Q of 0.25, and activation time of 30 ms. Glycan precursor ions were isolated for MSⁿ using a isolation width of 3.0 m/z. FTICR experiments were carried out by first isolating the precursor or fragment ion in the LTQ with a isolation width of 10 m/z then performing FTICR at 100,000 resolution. Quantitation was performed by separately adding the ¹³CH₃-labeled and ¹²CH₂D-labeled ion intensities over all isotopomers for each glycan.

Results and Discussion

Principle of Quantitation by Isobaric Labeling (QUIBL).

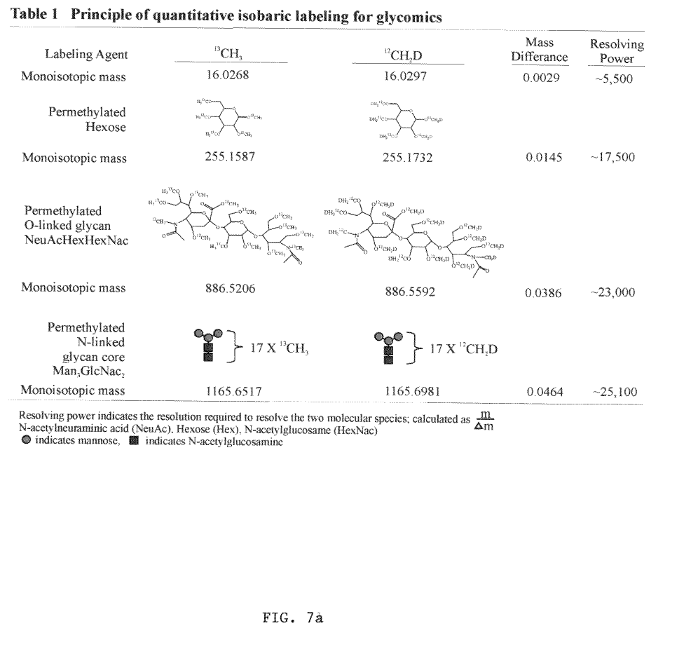

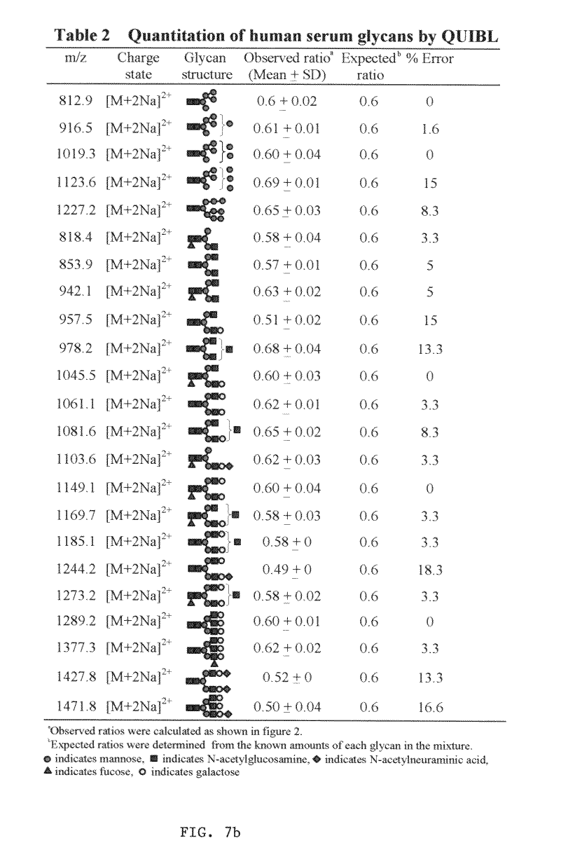

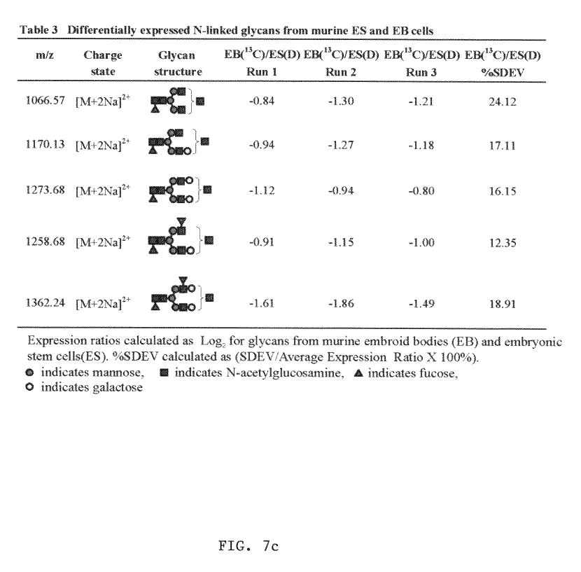

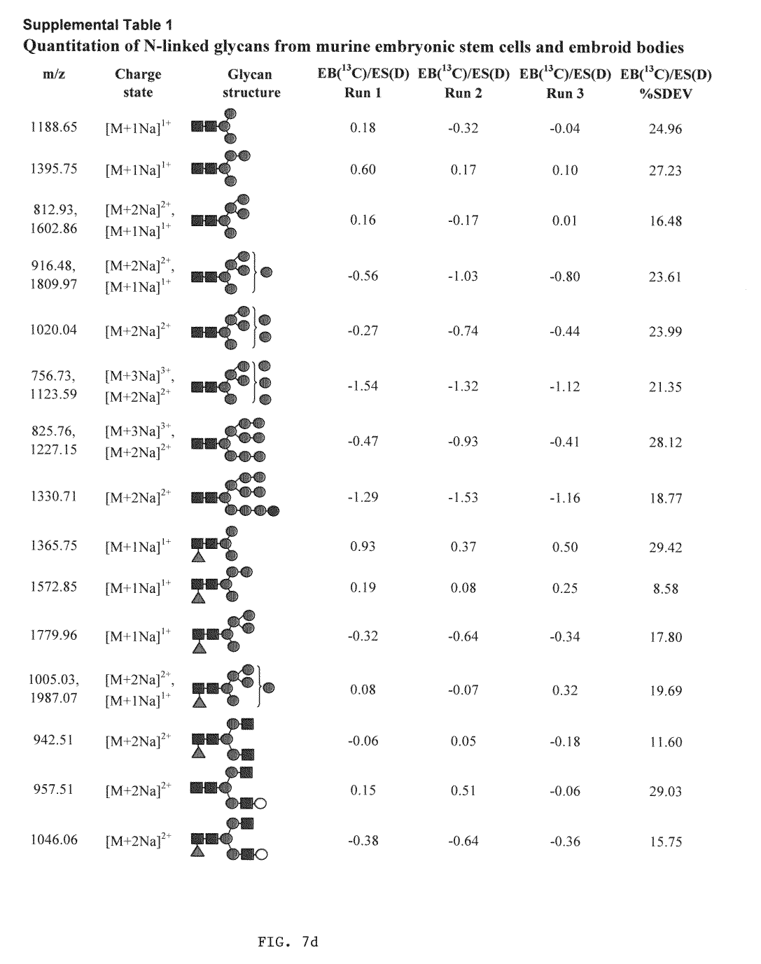

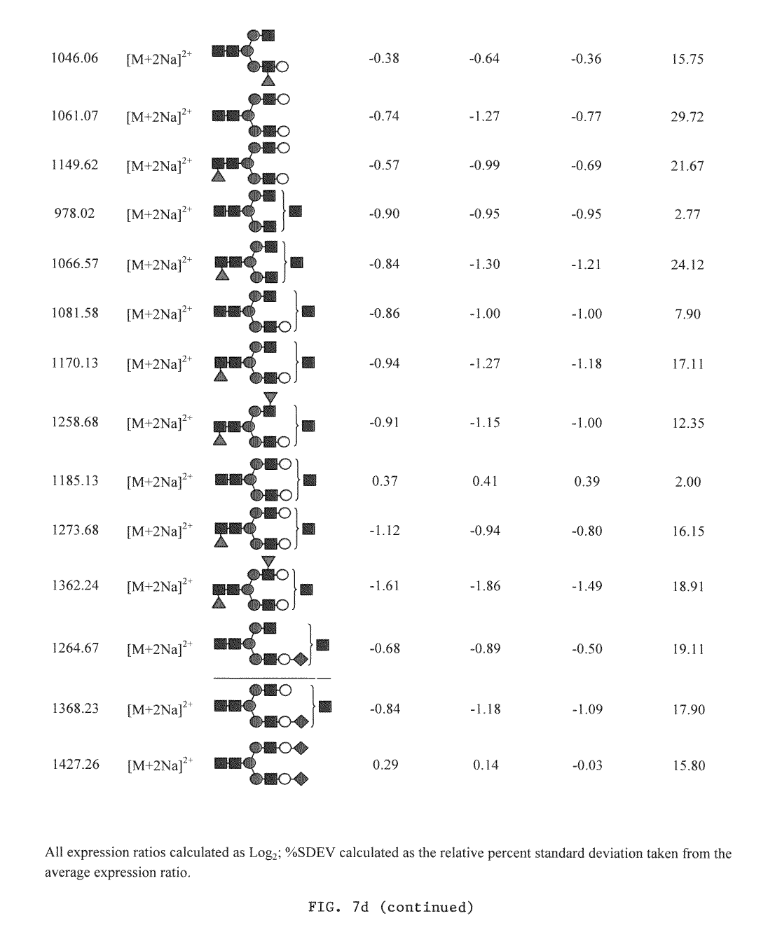

QUIBL involves the use of ¹³CH₃I or ¹²CH₂DI to generate isobaric pairs of per-O-methylated glycans. Two or more compounds are considered to be isobaric if they possess the same nominal mass (i.e., total number of protons and neutrons) but have different elemental or isotopic compositions. The exact masses of ¹³CH₃I and ¹²CH₂DI differ by 0.002922 Da, and thus isobaric analyte pairs containing a single label are difficult to resolve using current mass spectrometers. However, glycans which contain multiple methylation sites (i.e., —OH and NH₂ groups) are multiply labeled, increasing the □m between differentially labeled analytes and allowing them to be separated at a resolution of ˜30,000 m/μm. As the number of methylation sites increases, the mass difference for a pair of differentially labeled isobaric species and the total mass of the glycan also increase in parallel (Table 1 as shown in FIG. 7a). Hence, the resolution (m/□m) needed to resolve a pair of isobarically labeled glycans is practically independent of the glycan's molecular mass.

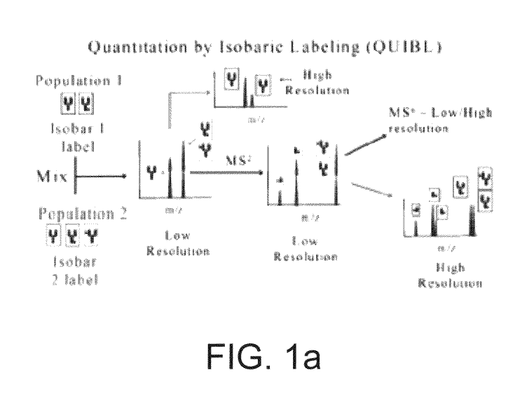

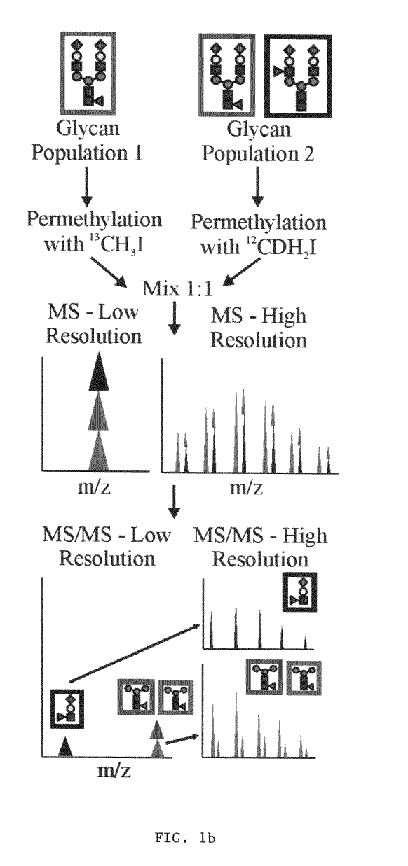

The QUIBL method consists of six steps (FIG. 1). (i) Two samples containing the same glycans in different proportions are permethylated with either ¹³CH₃I or ¹²CH₂DI. (ii) The permethylated samples are mixed (in equal ratios) and analyzed using a hybrid tandem mass spectrometer (such as an ion trap-Fourier transform ion cyclotron resonance mass spectrometer (FTICR) or an ion trap-orbitrap) capable of both low-resolution and high-resolution mass analysis. Nominal analyte masses are determined at low resolution using the ion trap, which is unable to resolve differentially labeled quasimolecular ions that are otherwise identical. (iv). Quasimolecular ions are analyzed (using the FTICR or orbitrap) at high resolution to distinguish ions originating from the ¹³CH₃ and ¹²CH₂D labeled glycans. Direct comparison of quasimolecular ion abundances in MS mode (without fragmentation) provides a measure of the abundance ratio for each glycan that is not a component of an isomeric mixture. Analysis of such mixtures, which contain glycans having the same elemental composition but different chemical structures, requires tandem MS. (v) The structures of quasimolecular precursor ions are identified by MSⁿ in the low resolution mass analyzer. At this stage, differentially labeled fragment ion pairs (which are otherwise identical) appear at the same nominal mass and thus the ion selection process does not discriminate between the isobaric labels. (vi) The resulting fragment ions are analyzed at high resolution and the abundance ratio for each isomer is determined by comparing ion abundances in a differentially labeled ion pair that is diagnostic for that particular isomer.

Standard Glycan Analysis using QUIBL

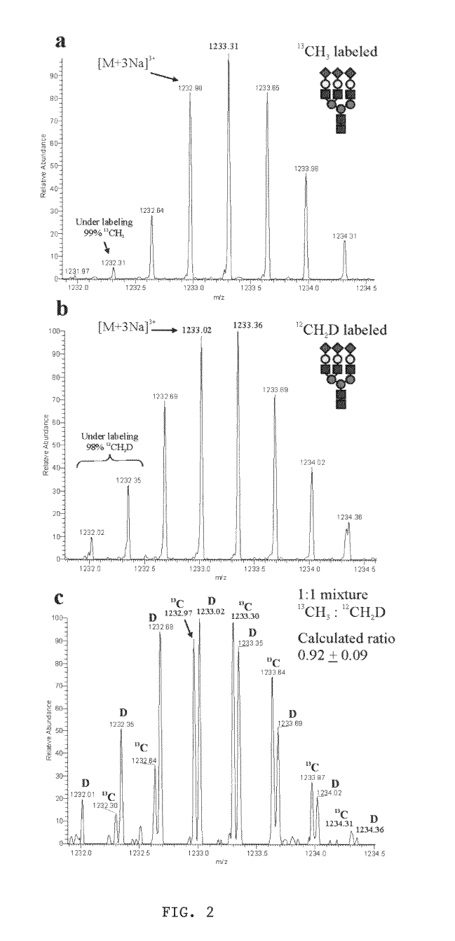

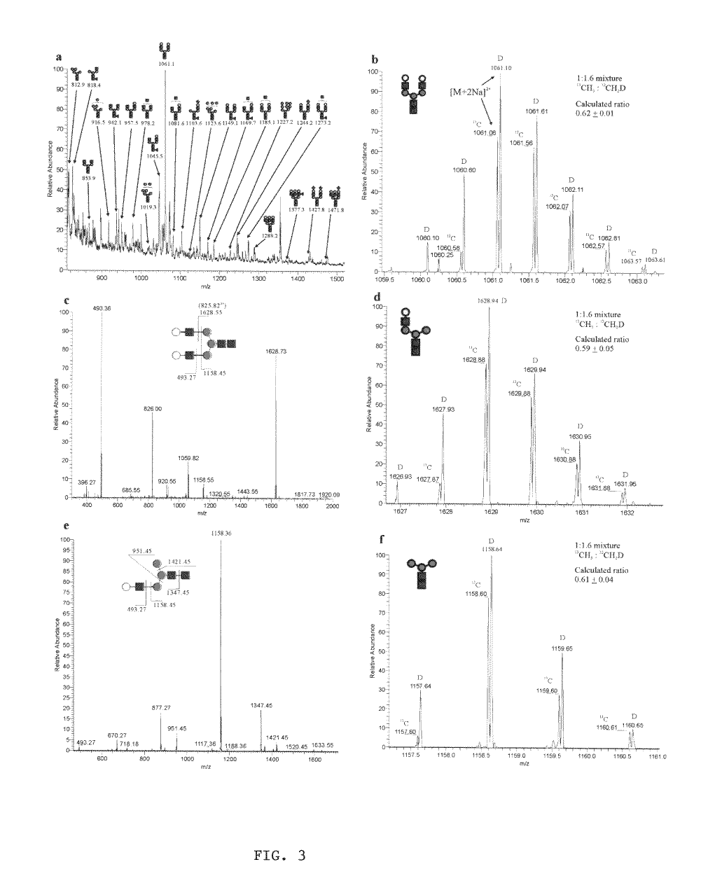

Two glycans purified from bovine fetuin were used as standards to demonstrate the principles of the QUIBL method. The FTICR spectra of the triantennary glycan from fetuin permethylated with ¹³CH₃I or ¹²CH₂DI are shown in FIGS. 2a and 2b, respectively. Each isotopic quasimolecular ion in the spectrum of the ¹²CH₂D labeled glycan is shifted in its mass-to-charge ratio (m/z) units by 0.05 compared to its ¹³CH₃ labeled counterpart, in good agreement with the shift predicted for the presence of 50 methyl groups on a triply charged ion ([0.0029×50]/3=0.05). It is noteworthy that the distribution of isotopic ion abundances depends on the label, as isotope ions at masses lower than the predicted monositopic mass have a higher abundance in the spectrum of the ¹²CH₂D labeled glycan than in spectrum of the ¹³CH₃ labeled glycan. This is due to the lower isotopic enrichment in ¹²CH₂DI, which contains 98% D, than in ¹³CH₃I, which is 99% ¹³C. For some traditional isotopic labeling procedures, the use of incompletely labeled reagents results in overlapping isotopic peaks, i.e., the ion produced by the under incorporated “heavy” species appears at an m/z value that is indistinguishable from an ion produced by the “light” species. In the QUIBL experiment, incompletely labeled ions are still resolved (FIG. 2c). Replacing one of the many ¹³C atoms with a ¹²C atom or replacing one of the many D (or ²H) atoms with an ¹H atom decreases the analyte's mass by approximately 1 Da, however, the resulting ion is detected in the appropriate (¹³CH₃-labeled or ¹²CH₂D-labeled) ion series because it still contains a large number of isotopic labels. This greatly simplifies quantitation, which is accomplished by summing the ion abundances for the ¹³CH₃-labeled and ¹²CH₂D-labeled series and comparing these two values. The average ratio obtained by applying this method to a standard 1:1 mixture of differentially labeled, triantennary fetuin glycan was 0.92±0.09 (FIG. 2c).

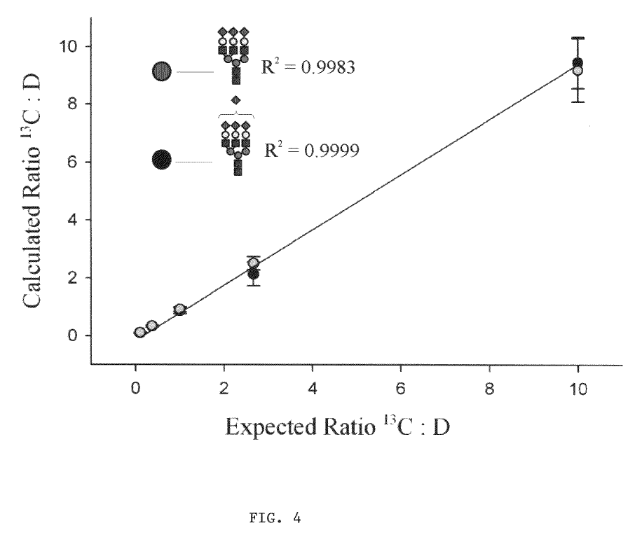

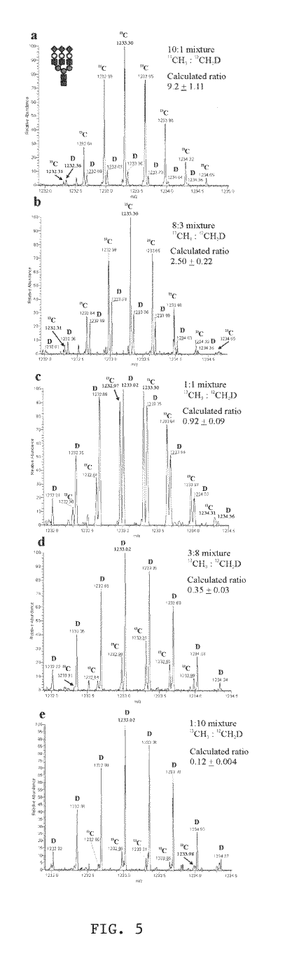

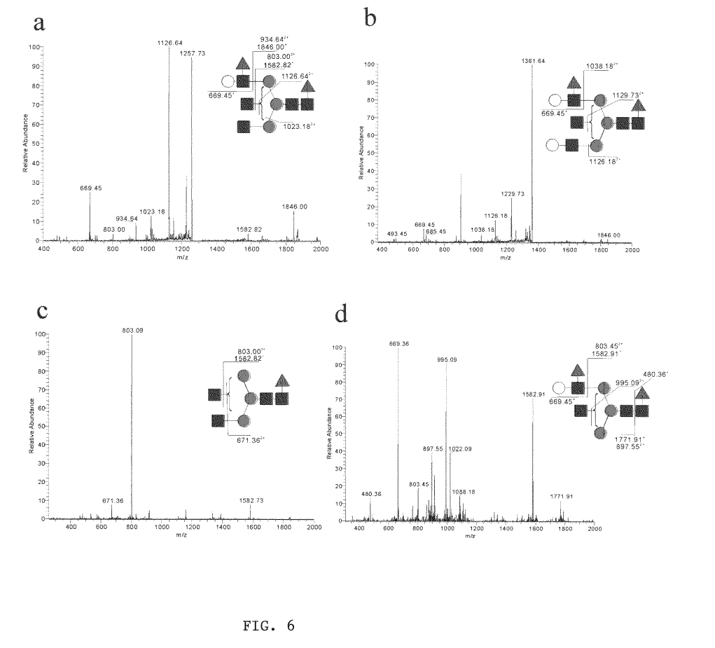

The linearity of response obtained by QUIBL was evaluated by FTICR analysis of five standard mixtures prepared by combining fetuin glycans labeled with ¹³CH₃ and ¹²CH₂D in ratios ranging from 10:1 to 1:10 (FIGS. 4 and 5). The analysis of two triantennary fetuin glycans (performed in triplicate) is shown in FIG. 4. These results indicate that quantitation using the QUIBL approach is linear over two orders of magnitude. The accuracy of the QUIBL method, as with other isotopic labeling methods increases as the ratio of two labeled species approaches one. This is illustrated in FIG. 5, which shows the high-resolution MS spectra of one of the fetuin glycans from the labeled mixtures. At ¹³CH₃ to CH₂D ratios of 1:1, 8:3 and 3:8, all isotopomer signals, including those due to under isotopic incorporation, are clearly visible and contribute to the accuracy and reproducibility of the ratio measurements. For these mixtures, the maximum error was below 17%, which is comparable to other quantitation methods utilizing isotopic labeling. However, as the ratio is increased to 10:1 or decreased to 1:10, the low abundance peaks become more difficult to discern, and the standard deviations and errors associated with the ratio measurements becomes larger.

Application of QUIBL to Human Serum Glycans

Serum glycomics is emerging as a potentially valuable method for the discovery and characterization of biomarkers for human diseases. To date, quantitative serum glycomics has been performed using isotopic labels that cause large mass shifts. These approaches have numerous drawbacks, including the doubling of sample complexity and the inability to quantitate individual isoforms. We therefore evaluated the QUIBL approach for its ability to quantitate glycans released from human serum. Serum glycans permethylated with either ¹³CH₃I or ¹²CH₂DI were mixed in a 1:1.6 ratio and analyzed in triplicate using an LTQ-FT (FIG. 3). MS was first performed using the low resolution LTQ to determine the nominal masses of the glycans (FIG. 3a). Individual glycans were identified through multiple rounds of collision induced dissociation (MSⁿ) (FIGS. 3c and 3e) and quantified by analysis of the fragment ions using the FTICR (FIGS. 3b, 3d, and 3f). For each glycan, the QUIBL method generates pairs of differentially labeled ions having the same nominal mass. This confers three distinct advantages to the QUIBL method compared to traditional isotopic labeling procedures. The first is an increase in ion abundance during low-resolution MS and MSⁿ, as both of the ions of a differentially labeled pair are detected at the same m/z. This factor reduces the amount of material needed for the glycan identification stage of the analysis. The second advantage is that glycans that are normally resolved by MS due to molecular weight differences are still resolved during QUIBL analysis. This is not true of traditional labeling, as the large mass shifts that are introduced by these methods often cause the light form of one glycan to have a mass that is very close to the mass of the heavy form of a completely different glycan. The resulting spectral overlap interferes with both identification and quantitation of the glycans. The third advantage is that the QUIBL method is not susceptible to errors arising from differences in detection efficiency that would occur if the differential labeling resulted in a large mass difference. The QUIBL approach accurately quantitated a broad range of glycan structures in a complex mixture in a single experiment (Table 2 as shown in FIG. 7b). These results demonstrate that QUIBL does not depend on glycan composition, size, or ionization efficiency, and is capable of accurately quantitating glycans of both low and high abundance. The maximum error in the calculated glycan ratios for the differentially labeled samples was 18.3% with an average error of 4.8%.

Perhaps the most promising aspect of QUIBL is that it allows simultaneous quantitation of glycans that have the same molecular mass (i.e., isomers). That is, if a fragment ion unique to each of the isomers is observed by MSⁿ, the ratio of differentially labeled forms of each isomer can be measured by high-resolution analysis of the fragment ions (FIGS. 3d and 3f). This capability was demonstrated by the selection and fragmentation of the [M+2Na]²⁺ ion (m/z 1061.1) of the serum glycan Man₃GlcNAc₄Gal₂ (FIG. 3c). CID of this precursor ion generated a collection of fragments that included a singly charged ion (m/z 1628.55), which was analyzed by high resolution FTICR (FIG. 3d). The isobaric labeling of this fragment ion was present at the same 1:1.6 ratio as observed for the intact precursor ion in FIG. 3b. The m/z 1628.73 fragment ion (FIG. 3c) was subjected to CID for MS³ analysis (FIG. 3e). Selection and FTICR analysis of the resulting MS³ fragment at m/z 1158.36 (FIG. 3f) gave the same ratio, demonstrating that accurate quantitation can be performed using fragment ions originating from multiple MS/MS events. These results suggest that QUIBL can be used for the accurate quantitation of glycans that are present as low abundance components of isomeric mixtures.

Application of QUIBL for Quantifying Glycome Changes During Early Embryogenesis.

Mammalian pluripotent embryonic stem cells (ESCs) are derived from the inner cell mass (ICM) of blastocyst-stage embryos. When cultured over extensive periods of time under appropriate conditions, ESCs retain many of the characteristics associated with pluripotent cells of the ICM, including the capacity to generate the three embryonic germ lineages (ectoderm, endoderm and mesoderm) and the extraembryonic tissues that support development. In murine ESCs, Leukemia inhibitory factor (LIF) stimulates the renewal of mouse ESCs and suppresses their differentiation. Removal of LIF from the media promotes the differentiation of ESCs into spheroid colonies called Embryoid Bodies (EBs), which recapitulate certain aspects of early embryogenesis such as the appearance of lineage-specific regions of differentiation. The pluripotency of ESCs provides the basis for developing a wide variety of somatic and extraembryonic tissue cultures with potential therapeutic applications in the treatment of diseases and injuries.

We applied QUIBL to compare N-linked glycan expression levels in murine ESCs and Embryoid Bodies (EBs). To quantify the changes in glycan expression that accompany differentiation of ESCs into EBs, we isolated N-linked glycans from 10⁷ cells of each type. The ESC glycans, labeled with ¹²CH₂DI, were mixed with the EB glycans labeled with ¹³CH₃I and the mixture was analyzed as described in FIG. 1. FIG. 1 illustrates a flow chart for quantitative glycan analysis using isobaric labeling. Glycans from two biological samples are permethylated in either ¹³CH₃I or ¹²CH₂DI and mixed together prior to analysis. At low mass resolution, the two labeled species appear at the same m/z value thereby increasing their abundance and decreasing sample complexity. Analysis of the glycans by high resolution MS separates the differentially labeled glycan precursor ions permitting their relative quantitation by comparing the peak intensities from the ¹³CH₃ to the ¹²CH₂D labeled glycans. Structural information on the glycan is provided by low resolution MSⁿ, which does not alter the ratio of isobaric labels. High resolution analysis of the MSⁿ fragment ions permits the isomeric glycans to be quantified.

In total, 29 distinct glycans, ranging from high mannose to complex triantennary forms, were characterized and quantitated (FIG. 7d, Table 4). This demonstrated the potential of QUIBL analysis to accurately quantitate a diverse population of glycans, as shown by an average relative standard deviation below 19% for the entire dataset.

Changes in the expression levels of several cell surface glycan markers, including SSEA1 (stage specific embryonic antigen 1, also known as Lewis X) and the Forssman antigen (FA), are associated with the differentiation of murine ESCs to EBs. Both of these markers are preferentially expressed in ESCs. During early development of the mouse embryo, the Lewis X antigen is expressed as part of embrioglycan, an O-linked proteoglycan that disappears during development. We observed that differentiation of ESC into EBs was accompanied by a greater than three-fold decrease in the expression of two di-fucosylated (Lewis X type) N-linked glycans (FIG. 7c, Table 3) whose structures were confirmed by MSⁿ analysis (FIG. 6). Thus, our results are consistent with previous reports describing a decrease in the expression of Lewis X when ES cells differentiate into EBs. We also observed a twofold decrease in the expression of several other complex fucosylated N-linked glycans. Notably, our results indicate that the developmental regulation of Lewis X epitope is expressed in N-linked glycans and not restricted to the polylactosamine O-linked structures of embryoglycan.

### CONCLUSION

Herein we have introduced a novel strategy, based on the use of isobaric labeling, for quantitative/comparative glycomics. The QUIBL method was successfully used to analyze N-linked oligosaccharides released from a standard glycoprotein and from human serum. Isobaric labeling was also used to identify changes in the glycoproteome associated with the transition of mouse embryonic stem cells to embryoid bodies. In this case, we were able to observe that N-linked glycans containing the Lewis X structure were more abundant in the ES cells than EB. There are numerous advantages of the QUIBL approach, many of which result from the isobaric ions appearing at the same nominal mass to charge ratio. This characteristic leads to increased ion intensity as ions from both samples are not distributed between isotopic species having different m/z values. The small mass difference between these isobars allows the two species to be simultaneously selected for MSⁿ analysis, permitting the relative quantitation of isomeric glycans, as was used to determine the increased expression of Lewis X glycans discussed above. Although the focus of this presentation is on glycoprotein glycans, this strategy is directly applicable to oligosaccharides from other sources, such as glycolipids. The concept of isobaric labeling is expected to be applicable to other types of “omics” analyses with other derivatizing agents. Lastly, we anticipate that isobaric labeling will also provide a manner to method allowing the absolute quantification of these molecules.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified. In addition, the phrase “about ‘x’ to ‘y’” includes “about ‘x’ to about ‘y’”. The term “consisting essentially of” is defined to include a formulation that includes the inks or dyes specifically mentioned as well as other components (e.g., solvents, salts, buffers, biocides, binders, an aqueous solution) using in an ink formulation, while not including other dyes or inks not specifically mentioned in the formulation.

Many variations and modifications may be made to the above-described embodiments. All such modifications and variations are intended to be included herein within the scope of this disclosure and protected by the following claims.

We claim:

1. A method of quantifying compounds, comprising:
labeling a first set of compounds with a first isobaric label;
labeling a second set of compounds with a second isobaric label;
mixing the first set of compounds with a second set of compounds to form a third set of compounds; and
analyzing the third set of compounds with a mass spectrometry system.

2. The method of claim 1, wherein the first compound and the second compound are each selected from glycans, glycoproteins, proteins, carbohydrates, or glycolipids.

3. The method of claim 1, wherein the first isobaric label and the second isobaric label each include elements that are used to form isobaric labels and these elements are selected from: 1/2H, 15/14 N, 13/12 C, 16/18 O, 32/34 S, 35/37 Cl, or 79/81 Br.

4. The method of claim 1, wherein the first isobaric label is ¹³CH₃ and the second isobaric label is CH₂D.

5. The method of claim 1, wherein the mass spectrometry system includes a hybrid ion trap-FTMS or an ion trap-orbitrap mass spectrometry system.

7. The method of claim 1, wherein labeling a first set of compounds includes permethylation of the compounds with ¹³CH₃I, and wherein labeling a second set of compounds includes permethylation of the compounds with CH₂DI.

8. A method of identifying compounds, comprising:
labeling a first set of glycans with a first isobaric label;
labeling a second set of glycans with a second isobaric label;
mixing the first set of glycans with a second set of glycans to form a third set of glycans; and
analyzing the third set of glycans with a mass spectrometry system.

9. The method of claim 8, wherein the first isobaric label and the second isobaric label each include elements that are used to form isobaric labels and these elements are selected from: 1/2H, 15/14 N, 13/12 C, 16/18 O, 32/34 S, 35/37 Cl, or 79/81 Br.

10. The method of claim 9, wherein the mass spectrometry system includes a hybrid ion trap-FTMS or an ion trap-orbitrap mass spectrometry system.

11. The method of claim 8, wherein the first isobaric label is ¹³CH₃ and the second isobaric label is CH₂D.

12. The method of claim 11, wherein the mass spectrometry system includes a hybrid ion trap-FTMS or an ion trap-orbitrap mass spectrometry system.

13. The method of claim 8, wherein labeling a first set of glycans includes permethylation of the glycans with ¹³CH₃I, and wherein labeling a second set of glycans includes permethylation of the glycan with CH₂DI.

14. The method of claim 13, wherein the mass spectrometry system includes a linear ion trap-Fourier transform hybrid mass spectrometry system.

15. The method of claim 13, wherein the mass spectrometry system includes a quadrupole-time of flight hybrid mass spectrometry system.

16. A method of quantifying compounds, comprising:
labeling a first set of compounds with a first isobaric label;
labeling a second set of compounds with a second isobaric label;
labeling at least one more set of compounds with at least one isobaric label, wherein each of the additional sets of compounds is labeled with a unique isobaric label;
mixing the first set of compounds, the second set of compounds, and the one or more sets of compounds to form a resultant set of compounds; and
analyzing the resultant set of compounds with a mass spectrometry system.

17. The method of claim 16, wherein the first isobaric label and the second isobaric label each include elements that are used to form isobaric labels and these elements are selected from: 1/2H. 15/14 N, 13/12 C, 16/18 O, 32/34 S, 35/37 Cl, or 79/81 Br.
